(19) United States
(12) Patent Application Publication
(10) Pub. No.: US 2012/0330271 A1
(43) Pub. Date: Dec. 27, 2012

(54) Method and Apparatus for the Containment of a Surgical Site

(76) Inventor: James E. Burgess, Gibsonia, PA (US)

(21) Appl. No.: 13/363,482

(22) Filed: Feb. 1, 2012

Related U.S. Application Data
(63) Continuation of application No. 11/939,248, filed on Nov. 13, 2007, now abandoned.
(60) Provisional application No. 60/858,570, filed on Nov. 13, 2006.

(51) Int. Cl. A61M 31/00

(52) U.S. Cl. 604/500; 604/93.01

(57) ABSTRACT

An apparatus for the containment of a surgical site includes an enclosure, with at least one port, securely coupled to the surgical site and a source of pressurized fluid in fluid communication with the enclosure via the at least one port providing pressurized fluid to the surgical site. The pressurized fluid establishes a hemostatic equilibrium within the enclosure by reducing blood egress through injuries in blood vessels at the surgical site thereby preventing bleeding at the surgical site. A method for the containment of a surgical site includes securely coupling an enclosure to a surgical site; fluidly coupling a source of pressurized fluid to the enclosure; and continuously providing pressurized fluid to the enclosure from the source of pressurized fluid and draining fluid from the enclosure. The pressurized fluid establishes a hemostatic equilibrium within the enclosure.

### CROSS REFERENCE TO RELATED APPLICATIONS

This application claims the benefit of U.S. Provisional Patent Application No. 60/858,570 entitled “Method and Apparatus for the Containment of a Surgical Site” filed Nov. 13, 2006, which is hereby incorporated by reference in its entirety.

### BACKGROUND OF THE INVENTION

1. Field of the Invention

The present invention relates, in general, to the containment of a surgical site and, more specifically, to the containment of a surgical site through the use of a fluid filled enclosure.

2. Description of Related Art

Intraoperative bleeding and its subsequent control are defining characteristics of most surgical procedures. The occurrence of unintended or uncontrollable hemorrhage during the course of a surgical procedure is an uncommon but dangerous event. Such hemorrhage may be due to a vessel wall defect, pathological vasculature, surgical misadventure, trauma or surgical release of a damaged vessel staunched by regional tissue pressure. Although most of these situations can be ultimately controlled, such hemorrhage may lead to serious complications. Specific situations include, but are not limited to, hemorrhage due to a vessel lesion such as a cerebral aneurysm, vessel rupture remote from a minimally invasive exposure, vessel rupture prior to adequate exposure, ruptured pathologic vessels such as an aortic aneurysm, an arteriovenous malformation, trauma, vascular tumors, etc. In these situations, blood flow interruption may result in tissue infarction or, in severe circumstances in which blood loss cannot be stopped, exsanguination and death.

Even non-fatal surgical hemorrhage has a variety of other negative impacts. For instance, after approximately a half liter of blood loss, intravascular hypovolemia begins to manifest as hypotension and tachycardia. This routinely necessitates volume replacement and occasionally blood product administration. Hemorrhage also obscures surgical anatomy. Accordingly, hemorrhage (or even the possibility of it) requires the surgeon's constant attention. Blood evacuation and intraoperative hemostasis routinely requires significant portions of the surgeon's time and attention, thereby diverting his or her attention from the actual procedure. While this prolongation of the procedure time is usually of little consequence, the obscuration of surgical anatomy due to innocuous bleeding may ultimately affect the efficiency and quality of the results.

During any surgical procedure, a patient will typically experience a slow, but steady, loss of blood through tissue blood vessel injuries through the normal course of dissection. While such blood loss does not pose the threat to the patient that surgical hemorrhage caused by a ruptured artery does, such blood loss may affect the efficiency of the surgeon because he or she must divert his or her attention from the surgical procedure. Furthermore, this slow seepage of blood into the surgical site obstructs the surgeon's view of the patient's anatomy thereby making the surgical procedure more difficult. Moreover, some blood loss has been unavoidable in most surgical procedures, and it is therefore easy to lose sight of the ideal of no blood loss.

Currently, a variety of methods are employed to control bleeding during surgical problems. The methods utilized are usually situation specific. In certain circumstances, low-level continuous hemorrhage may be allowed to continue during the surgical procedure, only to be staunched at the end. However, in most cases, efforts to maintain hemostasis (i.e., elimination of bleeding) are applied throughout the procedure. Accordingly, techniques have evolved within the various surgical disciplines to deal with surgical hemorrhage. For instance, manual compression, or tamponade, is an effective hemostatic maneuver. This method elevates the tissue pressure in the area of the hemorrhage thereby occluding the egress route of the blood. This method suffers from various drawbacks. For instance, this method also compromises healthy tissue perfusion so it is rarely useful as a permanent solution. Another method of minimizing bleeding and diminishing blood loss is the occlusion of the vascular pedicle of a hemorrhaging organ. However, this method leads to even greater perfusion compromise than tamponade does. Surgeons also implement microsurgical vessel repair to minimize bleeding when the injury is apparent and the exposure is sufficient. Surface cautery is another very popular means of controlling small vessel hemorrhage. Additionally, surgeons may use clamps and cauterization to minimize bleeding. However, each of these methods also suffers from various drawbacks.

Currently, there are instances in which orthopedic surgeons utilize uncontained pressurized fluid in certain surgical procedures. For instance, a joint can be infused with fluid in order to open the space for inspection and for manipulation. As an unintended side effect of such an application of fluid, it has been observed by orthopedic surgeons that bleeding is reduced due to the wound exposure to the fluid under pressure. However, in the course of an arthroscopic surgical procedure utilizing such fluid application, large quantities of saline solution tend to escape and typically run onto the operating room floor, notwithstanding attempts to collect the flow by using towels or plastic sheets draped in various ways. Depending on the type of operation and its duration, a large quantity of saline solution is lost. It is not uncommon to use between one to eight two-liter bags of saline solution during the course of a surgical procedure of this type.

Accordingly, although hemorrhage is a commonly encountered aspect of surgery, it diverts time and attention from the aim of the procedure and can occasionally be catastrophic. A need exists for an easy, safe and effective means to control and stop bleeding associated with surgical interventions.

### SUMMARY OF THE INVENTION

In order to meet this need, the present invention is a surgical site containment device and method that utilizes a contained fluid delivered under controlled, elevated pressure to an enclosure to obtain a hemostatic equilibrium at the surgical site. Such equilibrium is achieved when a fluid is delivered to a surgical site at a pressure that is elevated to the extent necessary to staunch flow out of rent, torn or otherwise opened blood vessels, veins, capillaries, arterioles, venules and arteries thereby minimizing or stopping bleeding at the surgical site.

The present invention is more particularly directed to an apparatus for the containment of a surgical site and to apply contained, pressurized fluid to the surgical wound, and allowing surgical manipulation to proceed through the container and the contained fluid. The apparatus includes an enclosure, with at least one port, securely coupled to the surgical site, and a source of pressurized fluid, in fluid communication with the enclosure via the at least one port providing pressurized fluid to the surgical site. The pressurized fluid establishes a hemostatic equilibrium within the enclosure by reducing blood egress through injuries in blood vessels at the surgical site, thereby minimizing or preventing bleeding at the surgical site.

The enclosure may include at least a first port, or a first port and a second port. The first port may be configured to be in communication with the source of pressurized fluid and the second port may be adapted to provide an outlet for the pressurized fluid. The enclosure may further include a third port adapted to receive a surgical tool therein and a fourth port adapted to receive optical devices therein. However, this is not to be construed as limiting the present invention as the number of ports corresponds to the number of entry points required by the tools necessary to perform the specific operation. If necessary, pressurized fluids (in and out) and all surgical tools may pass through a single port.

The enclosure may be constructed from aluminum, titanium or a polymer such as, but not limited to, polyethylene terephthalate, polyacrylate, polyurethane, or polycarbonate. At least a portion of the enclosure may be constructed from a transparent material thereby allowing a surgeon to see inside of the enclosure, or the enclosure may include a window formed therein to allow a surgeon to see inside the enclosure. The enclosure may be securely coupled to the surgical site via an adhesive, a pressure loaded device and/or gasket, a robotic arm or any combination thereof. The enclosure may further include at least one tool port. The at least one tool port may be sized to accommodate one or more surgical tools.

The pressurized fluid may be continuously delivered to the enclosure at a pressure of about 1 to 300 torr above ambient atmospheric pressure. The surgical site may be a patient's knee, abdomen, brain, spine, thoracic cavity, shoulder, elbow, wrist or any combination thereof.

The present invention is also directed to a method for the containment of a surgical site. The method includes the steps of securely coupling an enclosure to a surgical site; fluidly coupling a source of pressurized fluid to the enclosure; and continuously providing pressurized fluid to the enclosure from the source of pressurized fluid and draining fluid from the enclosure. The pressurized fluid establishes a hemostatic equilibrium within the enclosure by reducing blood egress through injuries in blood vessels at the surgical site, thereby preventing bleeding at the surgical site.

An alternative embodiment of the apparatus for containment of a surgical site may include an enclosure securely coupled to the surgical site, at least one sensor positioned within the enclosure for providing signals indicative of a condition within the enclosure, a source of pressurized fluid in fluid communication with the enclosure providing pressurized fluid to the surgical site and a control device electronically coupled to the source of pressurized fluid and the at least one sensor. The enclosure includes at least a first port adapted to provide an inlet for a pressurized fluid, a second port adapted to provide an outlet for fluids containing reusable blood cells and/or waste materials, a third port adapted to receive a surgical tool therein and a fourth port adapted to receive optical devices therein. The control device is configured to control the source of pressurized fluid based on signals provided by the at least one sensor. The pressurized fluid establishes a hemostatic equilibrium within the enclosure by reducing blood loss through injuries in blood vessels at the surgical site, thereby preventing bleeding at the surgical site.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. As used in the specification and the claims, the singular form of “a”, “an”, and “the” include plural referents unless the context clearly dictates otherwise.

### BRIEF DESCRIPTION OF THE DRAWINGS

FIG. 1 is a perspective view of an apparatus for the containment of a surgical site and a surgical site prior to coupling the apparatus to the surgical site in accordance with the present invention;

FIG. 2 is a perspective view of the apparatus for the containment of the surgical site after the apparatus has been securely coupled to the surgical site for the containment of the surgical site;

FIG. 3 is a cross-sectional view of the apparatus of FIG. 2;

FIG. 4 is a schematic diagram of the apparatus for the containment of the surgical site;

FIG. 5 is an alternative embodiment of the apparatus for the containment of the surgical site in accordance with the present invention;

FIG. 6 is a perspective view of the head of a patient having an open surgical site;

FIG. 7 is a perspective view of the head of a patient having another alternative embodiment of the apparatus for the containment of the surgical site coupled thereto; and

FIG. 8 is a perspective view of the head of a patient having the alternative embodiment of the apparatus for the containment of the surgical site of FIG. 7 coupled thereto after a flap of the skin has been positioned over the apparatus.

### DETAILED DESCRIPTION OF THE PRESENT INVENTION

For purposes of the description hereinafter, the terms “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

The present invention is directed to a method and apparatus for the containment of a surgical site. The method and apparatus utilizes pressure from a physiological fluid to establish a hemostatic equilibrium within an enclosure that surrounds the surgical site. A hemostatic equilibrium is achieved when the fluid pressure reduces blood loss caused by injuries in blood vessels at the surgical site thereby preventing bleeding at the surgical site. Injuries in blood vessels include, but are not limited to, tears, rents, or any other opening in a blood vessel. When such injuries occur, blood seeps into the surgical site. The application of a fluid pressure to these injuries that is slightly greater than the pressure of the blood seeping from the injury forces the blood to continue flowing through the blood vessel instead of flowing out of the injury. In this manner, a hemostatic equilibrium is established. The method and apparatus of the present invention advantageously allows the surgeon to perform a surgical procedure in a substantially blood free environment thereby enhancing clarity of the surgical site and allowing the surgeon to concentrate on the surgical procedure at hand rather than the cessation of bleeding.

Additionally, the apparatus of the present invention may be used both before and after a surgical procedure as well as during a surgical procedure. For instance, an emergency medical technician (EMT) may place the apparatus of the present invention over a wound of a patient in order to stop the wound from bleed until a surgeon can perform a surgical procedure. Furthermore, after a surgical procedure has been performed, the surgeon may leave the apparatus of the present invention on the surgical site for extended periods when no surgery is being performed to reduce swelling, maintain hemostasis, cleanse the surgical site or fight infection. Accordingly, as used herein, a surgical site is any open wound on any part of the body of a patient.

For purposes of illustration, the present invention will be described hereinafter in relation to a neurosurgical procedure. However, this is not to be construed as limiting the present invention, as the use of the present invention with any type of surgical procedure has been envisioned. For instance, the surgical site may be a patient's knee, abdomen, spine, thoracic cavity, shoulder, elbow, wrist or any other appropriate surgical site.

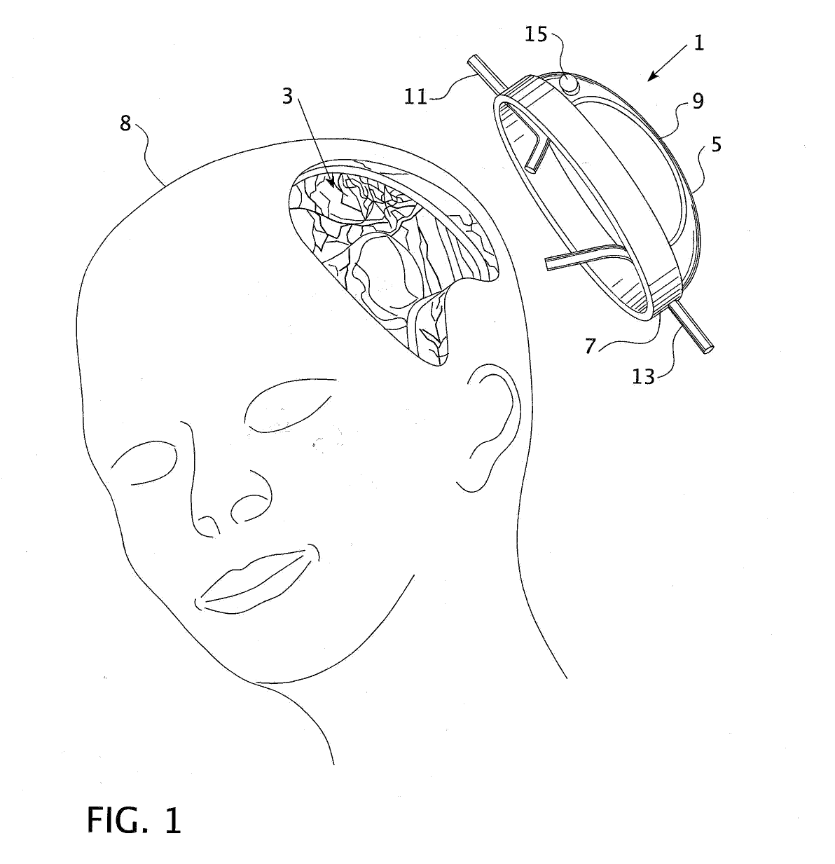

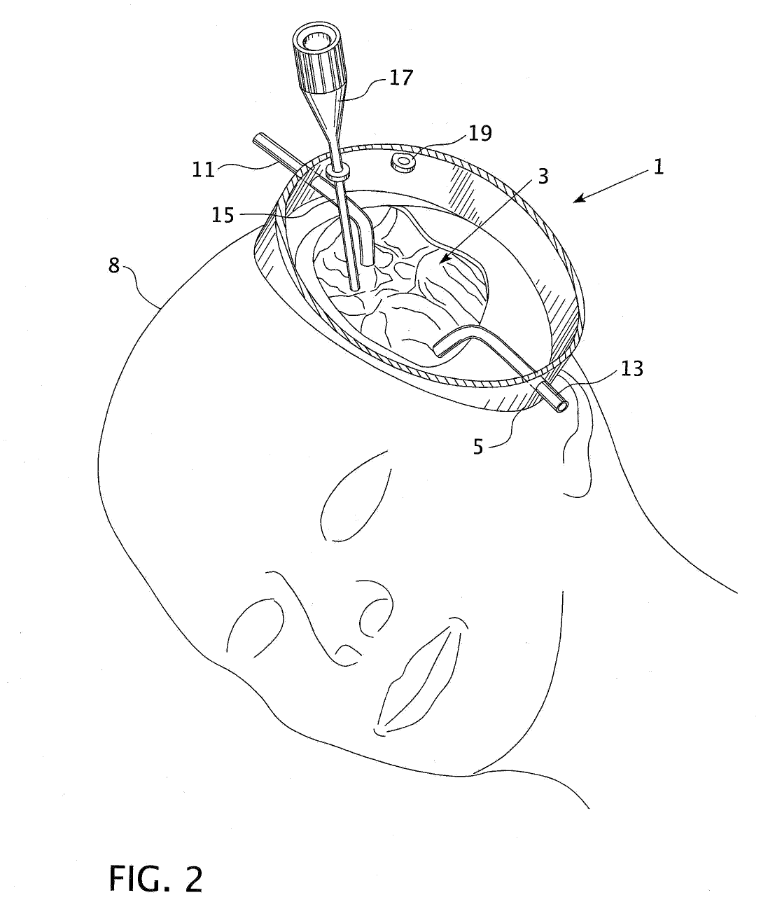

With reference to FIGS. 1-4, an apparatus 1 for the containment of a surgical site 3 includes an enclosure 5 with a base portion 7 configured to be securely coupled to the patient 8 around surgical site 3, and a viewing portion 9. Enclosure 5 may be constructed from any suitable material including, but not limited to, aluminum, titanium or a polymeric material such as, but not limited to, polyethylene terephthalate, polyacrylate, polyurethane, or polycarbonate, and may be rigid or flexible. Enclosure 5 may be constructed either entirely from a transparent material, may include a transparent view window or may be constructed entirely from an opaque material with a port for a camera, endoscope, microscope or a fiber optic imaging device. Enclosure 5 may also be any suitable shape and size depending on the surgical procedure that is being performed. For instance, enclosure 5 may be much larger if a surgery is being performed on a patient's back than if the surgery is being performed on a patient's head.

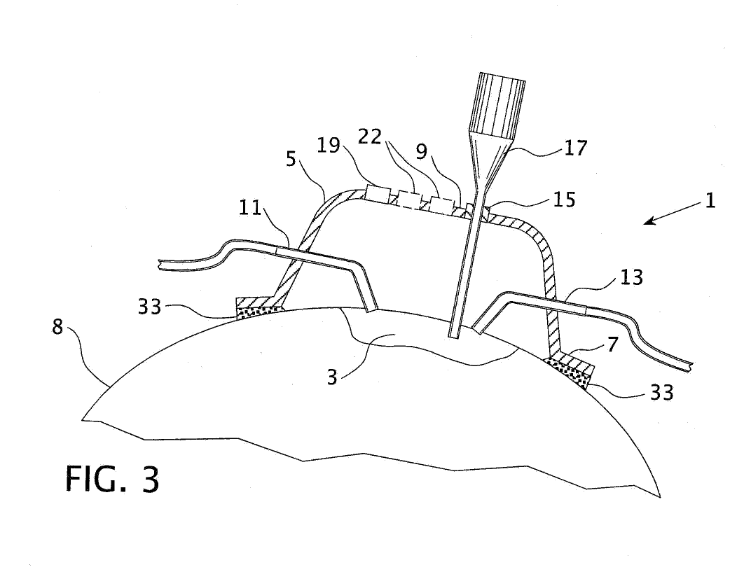

Enclosure 5 further includes a plurality of ports positioned thereon. For instance, enclosure 5 may include a first port 11 adapted to provide an inlet for a pressurized fluid, a second port 13 adapted to provide an outlet for fluids and/or waste materials, a third port 15 adapted to receive a surgical tool 17 therein and a fourth port 19 adapted to receive optical devices 21 therein. Optical devices 21 may include, but are not limited to, fiber optic imaging devices, cameras, lighting devices and the like. While enclosure device 5 was described herein above as including four ports, this is not to be construed as limiting as any appropriate number of ports have been envisioned. For instance, additional ports 22 (shown in phantom in FIG. 3) may be provided on enclosure 5 to allow access inside enclosure 5 for any tools necessary to perform a surgical procedure. Accordingly, the number of ports corresponds to the number of entry points required by the tools necessary to perform the specific operation. Furthermore, the size and shape of the ports may vary depending on the type of tool or instrument that is passing into enclosure 5. Additionally, the ports may be sized to accommodate one or more tools passing therethrough into enclosure 5.

Apparatus 1 also includes at least one sensor 23 coupled to enclosure 5 for providing signals indicative of a condition within enclosure 5, a fluid reservoir and pumping system 25 in fluid communication with enclosure 5 for providing pressurized fluid to surgical site 3 and a control unit 27 coupled to fluid reservoir and pumping system 25 and the at least one sensor 23.

The at least one sensor 23 may be any suitable device for determining the condition within enclosure 5. Sensor 23 may be as simple as a series of observations taken by the surgeon of the clarity of the fluid within enclosure 5 and control unit 27 may be a switch, foot pedal, dial or other suitable device for adjusting the pressure of the fluid provided by fluid reservoir and pumping system 25 to enclosure 5. Among the more complex embodiments, sensor 23 may be a colorimetric sensor, a pressure sensor and/or an oxygen saturation monitor, and control unit 27 may be a microprocessor or microcontroller. Control unit 27 may further include a fine manual control device (not shown). During the course of routine surgery, small vessel bleeding may occur that the surgeon wishes to immediately and permanently stop. The fine manual control device of control unit 27 provides the surgeon with the capacity to elevate the pressure just to the point of hemostasis in such cases.

In situations where a colorimetric sensor is used as sensor 23, the colorimetric sensor determines whether the fluid within enclosure 5 is any color other than clear. The fluid may have a color other than clear if it is mixed with blood from the wound opening. If the sensor determines that the fluid within enclosure 5 is colored, a signal is sent to control unit 27 to flush enclosure 5 with fluid from fluid reservoir and pumping system 25 until the colorimetric sensor indicates that the fluid within enclosure 5 is clear. The fluid from fluid reservoir and pumping system 25 enters enclosure 5 via first port 11 and the fluid mixed with blood and/or other waste materials exit through second port 13.

In situations where an oxygen saturation monitor is utilized as sensor 23, the oxygen saturation monitor is positioned in a portion of the patient's wound and determines the percentage of the patient's blood that is saturated with oxygen. Normally, the patient's blood should be 100% saturated with oxygen. When the pressure of the fluid provided at the wound site is too great, the pressure of the fluid will embarrass perfusion thereby preventing oxygen from reaching the tissue and blood vessels at the wound site. Such a condition may be harmful to the patient. Therefore, if the pressure of the fluid oxygen saturation of the patient's blood falls below a certain predetermined threshold level, such as 95%, the oxygen saturation monitor will send a signal to control unit 27 indicating that the pressure of the fluid being supplied to enclosure 5 should be decreased. Control unit 27 then decreases the pressure of the fluid either autonomously or under control of the surgeon by sending a signal to fluid reservoir and pumping system 25.

In situations where a pressure sensor is utilized as sensor 23, a pressure sensor is provided at the inlet of first port 11 and measures the ambient pressure of the fluid in the enclosure 5. A signal indicative of the pressure is then sent to control unit 27 by the pressure sensor. Control unit 27 may then display the pressure of the fluid on a display 29. The surgeon will then be able to monitor the pressure of the fluid by watching display 29 and adjust the pressure with a user interface (not shown). Display 29 may be any suitable display device such as, but not limited to, a cathode-ray tube display, a liquid crystal display, a plasma display or the like.

While the present invention has been described hereinabove as having a sensor 23 embodied as either a colorimetric sensor, a pressure sensor or an oxygen saturation monitor, this is not to be construed as limiting the present invention as the use of any other appropriate type of sensor or a combination of the above described sensors has been envisioned for use as sensor 23.

Fluid reservoir and pumping system 25 is designed to introduce a pressurized fluid to enclosure 5. The pumping system may be any suitable pumping device including, but not limited to, a manometric column, a pressure bag, a roller pump, a foot pump, a pulsatile pump or the like. The pressurized fluid is delivered from the fluid reservoir and pumping system 25 through standard medical tubing to first port 11 of enclosure 5. Excess fluid and waste are returned to a fluid reservoir through a piece of medical tubing fluidly coupled to second port 13 of enclosure 5. Prior to being returned to the fluid reservoir, the excess fluid and waste may be filtered by a filter 31 thereby allowing the fluid to be recirculated. Filter 31 may be any appropriate filter such as, but not limited to, a porous filter, a cyclonic filter or the like. Red blood cells may be harvested from the waste fluid and reused. Filtered waste fluid may be reused or discarded.

The fluid provided by fluid reservoir and pumping system 25 may be, but is not limited to, a physiological saline solution, synthetic cerebrospinal fluid (CSF), or the like. Antibiotic agents may be added to the fluid to prevent infection. Also, vocative agents, such as epinephrine, may also be added to the fluid to constrict blood vessels.

In operation, the area of the body where the surgery is to be performed is covered first with an antibacterial plastic adhesive sheet through which an incision is made thereby creating surgical site 3. Next, enclosure 5 is positioned over surgical site 3 and sealed to patient 8 via an adhesive 33. Adhesive 33 could also be a gasket of an intrinsically self-sealing and removable material such as, but not limited to a soft or elastic polymer. After the initial incision is made and enclosure 5 is securely coupled to surgical site 3, a pressurized fluid is pumped into enclosure 5 via first port 11 at an increasing pressure. The pressure at which the fluid is provided to enclosure 5 is desirably equivalent to the pressure of the blood at the ends of the exposed blood vessels, capillaries and arteries. The achievable pressure is from about 10 ton to about 300 torr. Very low pressures will stop minor bleeding from arterioles and capillaries. The fluid is continuously flushed from enclosure 5 until a clear environment is established in enclosure 5. Any waste materials and fluid mixed with blood are flushed from enclosure 5 via second port 13 as discussed hereinabove. The fluid is flushed from enclosure 5 and the environment within enclosure 5 clears. Hemostatic equilibrium is thereby established within enclosure 5 allowing surgical site 3 to remain blood free during the remainder of the procedure. The surgeon can then perform a surgical procedure using surgical tool 17 and any other tools. The pressure of the fluid being supplied to enclosure 5 is monitored using the at least one sensor 23, as discussed hereinabove, during the surgical procedure.

While the use of an adhesive was described hereinabove as securely coupling enclosure 5 to surgical site 3, this is not to be construed as limiting, as the use of any device for securely coupling enclosure 5 to surgical site 3 has been envisioned. For instance, a pressure loaded device, a robotic arm or temporary suturing may be used to securely couple enclosure 5 to surgical site 3.

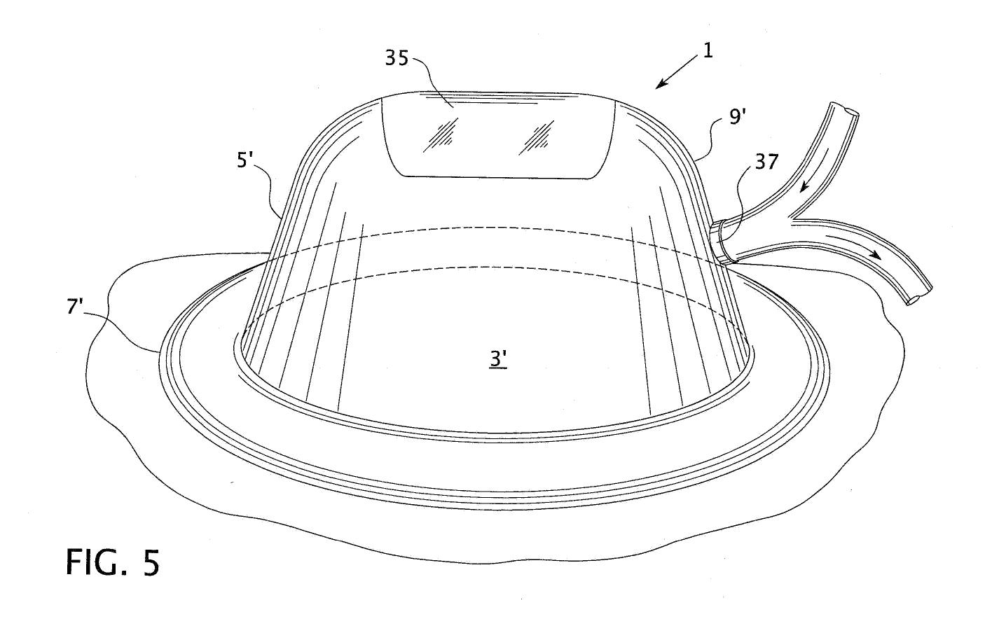

With reference to FIG. 5, an alternative embodiment of an apparatus for containing a surgical site is illustrated. This apparatus 1′ includes an enclosure 5′ with a base portion 7′ configured to be securely coupled to the patient around surgical site 3′, and a viewing portion 9′. Enclosure 5′ may be constructed from any suitable material including, but not limited to, aluminum, titanium or a polymeric material such as, but not limited to, polyethylene terephthalate, polyacrylate, polyurethane, and polycarbonate, and may be rigid or flexible. Enclosure 5′ may be constructed either entirely from a transparent material or may include a transparent view window 35. Enclosure 5′ may also be any suitable shape and size depending on the surgical procedure that is being performed. For instance, enclosure 5′ may be much larger if a surgery is being performed on a patient's back than if the surgery is being performed on a patient's head.

Enclosure 5′ further includes a single inlet/outlet port 37. Inlet/outlet port 37 allows fluid to enter enclosure 5′ and allows fluid to be flushed from enclosure 5′. This embodiment is desirable for situations where the patient has an open surgical site 3′ for extended periods of time and when the invention is utilized in the post-operative period. The patient may have an open surgical site covered by enclosure 5′ for extending periods when no surgery is being performed to reduce swelling, maintain hemostasis, cleanse the surgical site or fight infection. In instances where it is necessary to cleanse a surgical site, enclosure 5′ is irrigated at a higher flow than would generally be used during routine procedures. This cleansing could eliminate large clots, foreign material introduced during trauma, or spillage from intestinal injury in the abdomen. A measured amount of adjuvant material such as, but not limited to, antibiotics, surfactants, anticoagulants, etc. might be included in such cleansing procedures.

Another embodiment of the present invention utilizes an enclosure that is formed from a cavity of a patient's body. For instance, the concepts of the present invention may be utilized in laparoscopic surgeries. Laparoscopic surgery is a surgical technique for performing surgical procedures on the abdomen or pelvic cavity. Laparoscopic surgery includes making a first small incision on a patient's abdomen and inserting a Hopkins rod lens system connected to a video camera therein. A second small incision is made for a fiber optic cable system connected to a ‘cold’ light source, such as a halogen or xenon bulb, to illuminate the operative field. Additional incisions may be made to introduce surgical tools. In a conventional laparoscopic surgery, the abdomen is usually insufflated with carbon dioxide gas to create a working and viewing space. The present invention, rather than insufflating the abdominal cavity with carbon dioxide gas, provides a pressurized fluid to the abdominal cavity via an additional incision. The pressure of the fluid is increased until the surgeon views a cavity that is cleared. Excess fluid and waste fluid may be drained through the same incision or an additional incision.

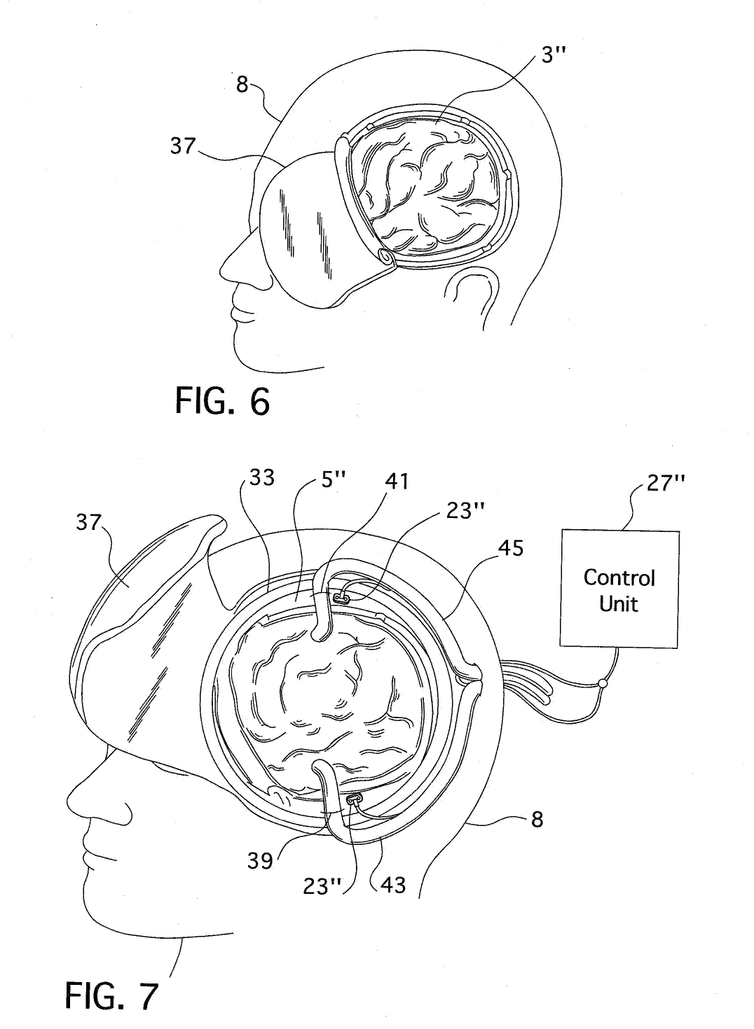

With reference to FIGS. 6-8, an additional embodiment of the present invention may be used during a decompressive craniectomy procedure. Decompressive craniectomy is a surgical procedure in which part of the skull is removed to allow a swelling brain room to expand without being squeezed. It is performed on victims of traumatic brain injury. As shown in FIG. 6, a decompressive craniectomy begins with the surgeon retracting a portion of the scalp 37 of a patient 8. Thereafter, a part of the skull is removed by the surgeon (not shown) to produce a surgical site 3″. The part of the skull that is removed is commonly referred to as a bone-flap.

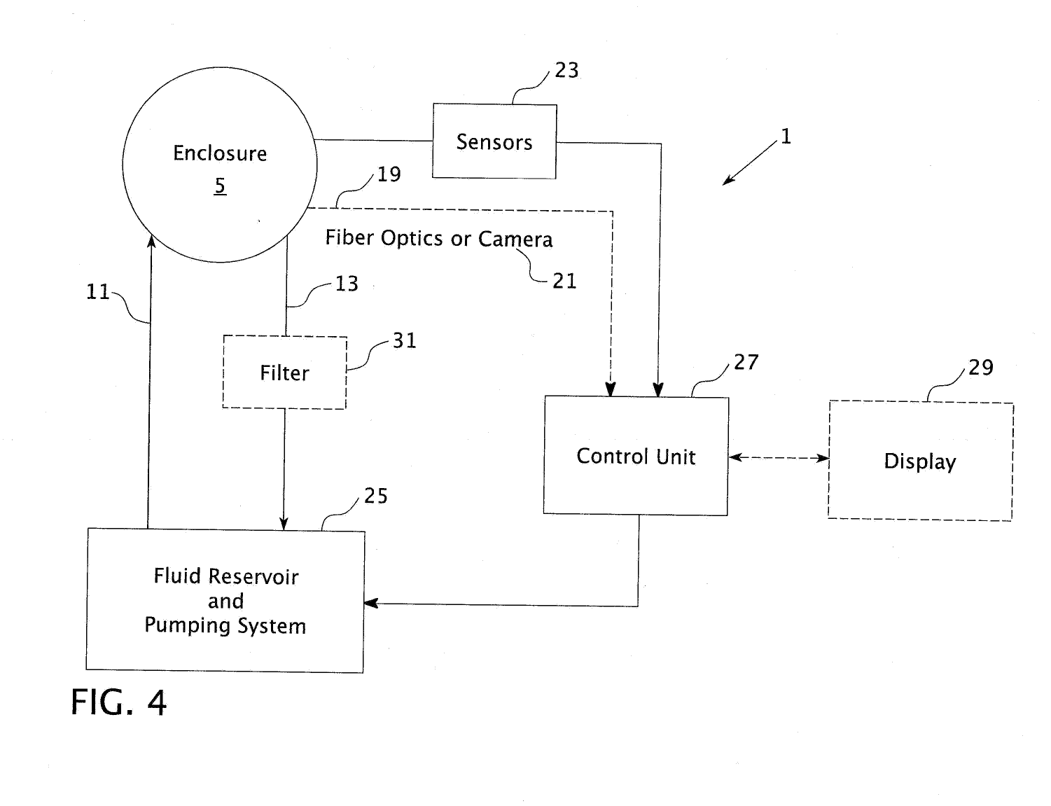

Thereafter, and with reference to FIG. 7, an enclosure 5″ of the apparatus for the containment of a surgical site 31 is positioned over surgical site 3″ and sealed to patient 8 via an adhesive 33 or any other suitable means. Enclosure 5″ is sized and shaped to correspond to the portion of scalp 37 of the patient 8 that has been retracted. Enclosure 5″ includes an inlet port 39 adapted to provide an inlet for a pressurized fluid and an outlet port 41 adapted to provide an outlet for fluids and/or waste materials. Inlet port 39 is coupled to a fluid conduit 43 for providing a pressurized fluid to surgical site 3″ and outlet port 41 is coupled to a fluid conduit 45 that provides an outlet for fluids and/or waste material. Fluid conduits 43, 45 are coupled to a fluid reservoir and pumping system (not shown) as discussed hereinabove with reference to FIG. 4. The apparatus further includes sensors 23″ positioned on enclosure 5″ and coupled to a control unit 27″ for monitoring enclosure 5″ as discussed hereinabove. Sensors 23″ may be, but are not limited to, colorimetric sensors, pressure sensors or oxygen saturation monitors.

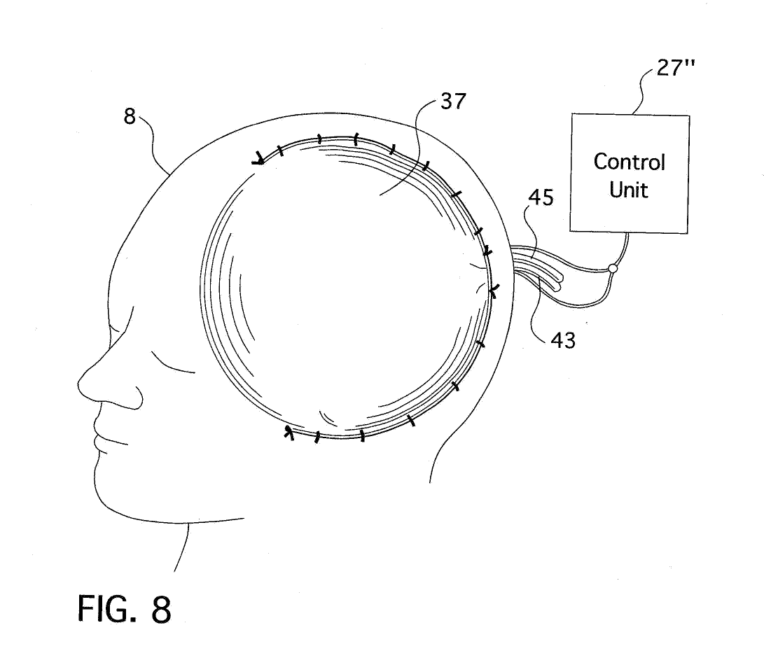

Enclosure 5″ is configured to stay on surgical site 3″ chronically until brain swelling has been reduced. Accordingly, and with reference to FIG. 8, once enclosure 5″ is properly coupled to surgical site 3″ and fluids are properly provided to and removed from enclosure 5″ via inlet port 39 and outlet port 41, respectively, the surgeon may position the portion of scalp 37 that has been retracted and suture the portion of scalp 37 back to the head of patient 8 thereby completely covering enclosure 5″ of the apparatus. The fluids provided to enclosure 5″ wash out cells and necrotic factors from surgical site 3″. In addition, the fluid provides modest hemostatic functions.

In addition to enclosure 5″, enclosure 5 as described in FIGS. 1-3 may also be used for a decompressive craniectomy by sealing third port 15 and fourth port 19.

A further variation on the disclosure, above, includes making the location of one or more access ports flexible and/or adaptable in any way feasible. For example, the large dome structure of FIG. 5 may be made from a flexible material, rather than a rigid material, so that any access means or one or more ports in the dome can be moved by the surgeon (or other health care practitioner) to a desired location without being rigidly constrained to a particular location or orientation in the overall device. Clearly, a surgeon has to have access to the surgical site within the present device with minimal or no tool positioning constraint. Additional variations, along this same port location flexibility theme, can include, without limitation, movable grids, expansible or stretchable ports or zipper-like or track-like constructs which allow the containment area to be customized on the spot as to the insertion location for one or more tools via one or more ports. It should be noted that in all embodiments of the invention but especially in any embodiment including one or more elastomeric or otherwise moveable or expansible/contractile walls, it is important to have in place an appropriate feedback control mechanism to maintain fluid pressure at the desired level to counteract the otherwise inevitable pressure changes which such elastomericity and/or movement would create.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements. Furthermore, it is to be understood that the present invention contemplates that, to the extent possible, one or more features of any embodiment can be combined with one or more features of any other embodiment.

The invention claimed is:

1. An apparatus for the containment of a surgical site comprising:
an enclosure, with at least one port, securely coupled to the surgical site; and
a source of pressurized fluid in fluid communication with the enclosure via the at least one port providing pressurized fluid to the surgical site;
wherein the pressurized fluid establishes a hemostatic equilibrium within the enclosure by reducing blood egress through injuries in blood vessels at the surgical site thereby preventing bleeding at the surgical site.

2. The apparatus of claim 1, wherein the enclosure includes at least a first port and a second port.

3. The apparatus of claim 2, wherein the first port is configured to be in communication with the source of pressurized fluid and the second port is adapted to provide an outlet for the pressurized fluid.

4. The apparatus of claim 2, wherein the enclosure further includes a third port adapted to receive a surgical tool therein and a fourth port adapted to receive optical devices therein.

5. The apparatus of claim 1, wherein the enclosure is constructed from aluminum, titanium, polyethylene terephthalate, polyacrylate, polyurethane, polycarbonate or any combination thereof.

6. The apparatus of claim 1, wherein at least a portion of the enclosure is constructed from a transparent material thereby allowing a surgeon to see inside of the enclosure.

7. The apparatus of claim 1, wherein the enclosure comprises a window formed thereon allowing a surgeon to see inside the enclosure.

8. The apparatus of claim 1, wherein the enclosure is securely coupled to the surgical site via an adhesive, a pressure loaded device, a robotic arm or any combination thereof.

9. The apparatus of claim 1, wherein the pressurized fluid is continuously delivered to the enclosure at a pressure of about 1 torr to 300 torr above ambient atmospheric pressure.

10. The apparatus of claim 1, wherein the surgical site is a patient's knee, abdomen, brain, spine, thoracic cavity, shoulder, elbow, wrist or any combination thereof.

11. The apparatus of claim 1, wherein the enclosure further comprises at least one tool port.

12. The apparatus of claims 11, wherein the at least one tool port is sized to accommodate one or more surgical tools.

13. A method for the containment of a surgical site comprising the steps of:
a) securely coupling an enclosure to a surgical site;
b) fluidly coupling a source of pressurized fluid to the enclosure; and
c) continuously providing pressurized fluid to the enclosure from the source of pressurized fluid and draining fluid from the enclosure,
wherein the pressurized fluid establishes a hemostatic equilibrium within the enclosure by reducing blood egress through injuries in blood vessels at the surgical site thereby preventing bleeding at the surgical site.

14. The apparatus of claim 13, wherein the enclosure includes at least a first port and a second port.

15. The method of claim 14, wherein the first port is configured to be in communication with the source of pressurized fluid and the second port is adapted to provide an outlet for the pressurized fluid.

16. The method of claim 14, wherein the enclosure further includes a third port adapted to receive a surgical tool therein and a fourth port adapted to receive optical devices therein.

17. The method of claim 13, wherein the enclosure is constructed from aluminum, titanium, polyethylene terephthalate, polyacrylate, polyurethane, polycarbonate or any combination thereof.

18. The method of claim 13, wherein at least a portion of the enclosure is constructed from a transparent material thereby allowing a surgeon to see inside of the enclosure.

19. The method of claim 13, wherein the enclosure is securely coupled to the surgical site via an adhesive, a pressure loaded device, a robotic arm or any combination thereof.

20. The method of claim 13, wherein the pressurized fluid is continuously delivered to the enclosure at a pressure of about 1 torr to 300 ton above ambient atmospheric pressure.

21. The method of claim 13, wherein the surgical site is a patient's knee, abdomen, brain, spine, thoracic cavity, shoulder, elbow, wrist or any combination thereof.

22. An apparatus for the containment of a surgical site comprising:
a) an enclosure securely coupled to the surgical site, the enclosure comprising at least:
i) a first port adapted to provide an inlet for a pressurized fluid;
ii) a second port adapted to provide an outlet for fluids and/or waste materials;
iii) a third port adapted to receive a surgical tool therein; and
iv) a fourth port adapted to receive optical devices therein; and
b) at least one sensor positioned within the enclosure for providing signals indicative of a condition within the enclosure;
c) a source of pressurized fluid in fluid communication with the enclosure via the first port, the source of pressurized fluid thereby providing pressurized fluid to the surgical site; and
d) a control device electronically coupled to the source of pressurized fluid and the at least one sensor, the control device configured to control the source of pressurized fluid based on signals provided by the at least one sensor,
wherein the pressurized fluid establishes a hemostatic equilibrium within the enclosure by reducing blood loss through injuries in blood vessels at the surgical site thereby preventing bleeding at the surgical site.
